(19) United States
(12) Patent Application Publication
(10) Pub. No.: US 2023/0355760 A1
(43) Pub. Date: Nov. 9, 2023

(54) MODIFIED CELLS OF LEUKEMIC ORIGIN AND A PD-L1 ANTIBODY FOR ENHANCING THE EFFICACY OF CANCER CELL THERAPY

(71) Applicant: Mendus B.V., Leiden (NL)

(72) Inventors: Erik Hans Manting, Leiden (NL); Satwinder Kaur Singh, Leiden (NL); Alex Karlsson-Parra, Leiden (NL); Haoxiao Zuo, Leiden (NL)

(21) Appl. No.: 18/119,487

(22) Filed: Mar. 9, 2023

Related U.S. Application Data
(60) Provisional application No. 63/318,484, filed on Mar. 10, 2022.

(51) Int. Cl. A61P 35/00; A61K 39/395; A61K 39/00; C12N 5/0783

(52) U.S. Cl. CPC A61K 39/4613; A61K 39/3955; A61K 39/464499; A61P 35/00; C12N 5/0646; A61K 2239/26; A61K 2239/28; A61K 2239/31; C12N 2502/30

(57) ABSTRACT

Composition and methods for ex vivo expansion of natural killer (NK) cells, and methods for cell-based cancer immunotherapy are disclosed. Leukemic cell-derived dendritic cells and anti-PD-L1 antibodies, and certain embodiments with addition of PBMCs are used for in vivo administration for cancer treatment. Leukemic cell-derived dendritic cells and anti-PD-L1 antibodies are also used for ex vivo expansion of NK cells.

### RELATED APPLICATIONS

This application claims the benefit of U.S. Provisional Application No. 63/318,484, filed Mar. 10, 2022, which is incorporated herein by reference in its entirety.

### BACKGROUND

Immune checkpoint blockade (ICB) therapies based on the administration of antibodies against the T cell inhibitory molecules cytotoxic T lymphocyte-associated protein 4 (CTLA-4) and programmed cell death 1 (PD-1, or its ligands) have been approved for the treatment of a variety of malignancies alone or in combination. However, in most tumors, ICB benefits a minority of patients, and only a subset of responders exhibits durable responses. To better understand the underlying mechanisms driving these variations in response, substantial work has been carried out to identify features of the tumor microenvironment (TME) that correlate with long-term survival and predict responses to ICB. One factor associated with outcome is the number of infiltrating T cells prior to or during early treatment, which introduces the concept of “hot” (inflamed and highly infiltrated) and “cold” (immunosuppressive and non-infiltrated) tumors. Patients who respond to ICB will most often exhibit a “hot” tumor phenotype.

These findings have led to an intensive search for combination treatments that could convert “cold” tumors into the “hot” lymphocyte-infiltrated state. There is now mounting evidence suggesting treatments that recruit and activate innate immune effector cells, including intratumoral NK cells and macrophages, can rapidly alter the TME via induction of tumor cell death and inflammation of the microenvironment, thus forming a feed-forward loop to boost T cell recruitment, function, and proliferation (Corrales et al, 2015; Salmon et al,,2016; Wang et al, 2021).

To date, a variety of immunotherapeutic strategies have been studied that provide some level of benefit when combined with ICB treatment in preclinical models or small clinical studies. However, in general the synergies discovered have been modest and the immunological ruleset to promote ICB responsiveness remains poorly defined. Moreover, in case of hematological malignancies, the novel immunotherapies such as ICB and CAR-T cells have not shown striking results as has been observed in solid tumors and lymphomas, respectively. More and more evidence has supported to utilize NK cells to harness the anti-tumor immune response in a wide range of malignancies, most notably with early evidence of efficacy in hematologic malignancies. Due to prior treatments such as chemotherapy, radiation, and immunosuppressants used in initial therapy and hematopoietic stem cell transplantation, patients with hematologic malignancies have low numbers of NK cells and are mostly dysfunctional. Restoring this innate immune deficit may lead to improved therapeutic outcomes as NK are specialized effector cells with innate ability to eliminate tumor cells.

### SUMMARY

The present disclosure provides methods and composition to address the above-described problems and is based, at least in part, on the finding that certain cells of leukemic origin can improve the expansion, efficacy and/or functionality of certain immune cells, particularly NK cells, when the cells of leukemic origin are mixed along with an anti-PD-L1 antibody, with allogeneic peripheral blood mononuclear cells (PBMCs)

In certain embodiments, the present disclosure provides a composition comprising a modified cell of leukemic origin, and an anti-PD-L1 antibody, wherein the modified cell exhibits a mature dendritic cell phenotype. In one aspect, modified cells that exhibits a mature dendritic cell phenotype are derived from a leukemia cell line, not isolated fresh from a healthy human subject. In another aspect, the composition further include a plurality of PBMCs, including natural killer (NK) cells. In one aspect, the PBMCs are derived from a patient. In another aspect, isolated NK cells are derived from allogenic PBMCs or from a NK tumor cell line, cord blood stem cells or from induced pluripotent stem cells.

In one embodiment, the cells of leukemic origin and an anti-PD-L1 antibody may be mixed with isolated NK cells and expand the NK cells ex vivo. In another embodiment, immune cells that are exposed to an anti-PD-L1 antibody and the modified cells of leukemic origin in vivo exhibit improved expansion, persistence, efficacy and/or functionality following administration to a patient by adoptive cell transfer.

In certain embodiments, the anti-PD-L1 antibody may bind to PD-L1 expressed on mature DCs. On the other hand, both the Fc domain of the anti-PD-L1 antibody and the mature DCs may interact with FcγR-expressing NK cells and myeloid mononuclear cells and enhance NK cell and myeloid mononuclear cell activation. In one aspect, this effect on FcγR-expressing immune cells occurs without addition of exogenous activation- or expansion-supporting cytokines such as IL-2, or IL15.

In certain embodiments, the modified cell and the anti-PD-L1 antibody together have a synergistic effect on activating FcγR-expressing immune cells. In one aspect, the immune cells are NK cells, and the modified cell and the anti-PD-L1 antibody together leads to between 5-fold to 80-fold expansion of NK cells with memory-like phenotype. In another aspect, the activation of the NK cells results in increased NK-mediated lysis of tumor cells. In another aspect, the activation of FcyR expressing immune cells, including myeloid mononuclear cells and NK cells, results in increased secretion of chemokines, inflammatory and effector cytokines.

In one aspect, the modified cell comprises at least one tumor antigen selected from the group consisting of WT-1, RHAMM, PRAME, MUC-1, p53, and Survivin. In another aspect, the modified cell is CD34-positive, CD1a-positive, CD83-positive, and CD14-negative. In another aspect, the modified cell further comprises a cell surface marker selected from the group consisting of DC-SIGN, Langerin, CD40, CD70, CD80, CD86, and any combination thereof. In another aspect, the modified cell is CD70-positive, CD80-positive, and CD86-positive.

In certain embodiments the modified cell comprises a costimulatory molecule. In certain exemplary embodiments, the costimulatory molecule is CD70.

In certain exemplary embodiments, the modified cell comprises an MHC class I molecule. In certain exemplary embodiments, the modified cell comprises an MHC class II molecule. In certain exemplary embodiments, the modified cell is non-proliferating.

In certain exemplary embodiments, the modified cell comprises a genetic aberration between chromosome 11p15.5 to 11p12. In certain exemplary embodiments, the genetic aberration encompasses about 16 Mb of genomic regions. In certain exemplary embodiments, the modified cell has been irradiated.

In certain embodiments, the anti-PD-L1 antibody has an intact Fc region and binds to Fc-gamma-receptors (FcγR), including FcγRI (CD64), FcγRIIA(CD32) and FcγRIII (CD16) with high affinity.

In certain embodiments, the anti-PD-L1 antibody is a mono-specific, bispecific or multi-specific antibody. Examples of an anti-PD-L1 antibody include but are not limited to avelumab or PDL-GEX.

In certain embodiments, a method for activating, stimulating and and/or expanding a population of immune cells is disclosed. In one aspect, the method includes: (a) obtaining a population of cells comprising immune cells, (b) contacting the population of cells with a modified cell of leukemic origin and an anti-PD-L1 antibody, wherein the modified cell exhibits a mature dendritic cell phenotype, and (c) co-culturing the population of cells and the modified cell of leukemic origin and the anti-PD-L1 antibody under conditions suitable to stimulate proliferation of the immune cells, thereby activating and expanding the population of immune cells.

In one aspect, the modified cell of leukemic origin for use in any of the methods disclosed herein comprises at least one tumor antigen selected from the group consisting of WT-1, RHAMM, PRAME, MUC-1, p53, and Survivin. In another aspect, the modified cell of leukemic origin is CD34-positive, CD1a-positive, CD83-positive, and CD14-negative. In another aspect, the modified cell further comprises a cell surface marker selected from the group consisting of DC-SIGN, Langerin, CD40, CD70, CD80, CD86, and any combination thereof. In another aspect, the modified cell is CD70-positive, CD80-positive, and CD86-positive. In another aspect, the modified cell of leukemic origin for use in any of the methods disclosed herein comprises an MHC class I molecule. In another aspect, the modified cell comprises an MHC class II molecule.

In other aspects, a method is provided for enhancing the efficacy of an adoptive cell therapy in a subject, comprising administering to the subject a composition comprising a modified cell of leukemic origin and an anti-PD-L1 antibody, wherein the modified cell comprises a mature dendritic cell phenotype and is non-proliferating, and wherein the subject has been administered an adoptive cell therapy.

In certain exemplary embodiments, the composition is administered to the subject about one day to about six months after the subject has been administered the adoptive cell therapy. In certain exemplary embodiments, the composition is administered to the subject about two days to about 21 days after the subject has been administered the adoptive cell therapy. In certain exemplary embodiments, the composition is co-administered to the subject with the adoptive cell therapy. In another exemplary embodiment, and disclosed composition is provided for enhancing the efficacy of systemic treatment with immune checkpoint blockers including anti-PD-1, anti-PD-L1 or anti-CTLA4

In other aspects, a method is provided for treating a disease or disorder in a subject, comprising: administering to the subject a composition comprising a modified cell of leukemic origin and an anti-PD-L1 antibody, wherein the modified cell comprises a mature dendritic cell phenotype and is non-proliferating; and administering to the subject an adoptive cell therapy.

In certain exemplary embodiments, the disease or disorder is a cancer. In certain exemplary embodiments, the cancer is a tumor. In certain exemplary embodiments, the tumor is a semi-solid tumor, including lymphomas. In certain exemplary embodiments, the tumor is a solid tumor. In certain exemplary embodiments, the cancer is liquid tumor, including acute myeloid leukemia (AML).

In other aspects, a composition is provided comprising ex vivo expanded immune cells, comprising a population of immune cells, a modified cell of leukemic origin and an anti-PD-L1 antibody, wherein the modified cell exhibits a mature dendritic cell phenotype.

In certain exemplary embodiments, the modified cell of leukemic origin comprises at least one tumor antigen selected from the group consisting of WT-1, RHAMM, PRAME, MUC-1, p53, and Survivin.

In certain exemplary embodiments, the modified cell of leukemic origin is CD34-positive, CD1a-positive, CD83-positive, and CD14-negative.

In certain exemplary embodiments, the modified cell of leukemic origin further comprises a cell surface marker selected from the group consisting of DC-SIGN, Langerin, CD40, CD70, CD80, CD86, and any combination thereof.

In certain exemplary embodiments, the modified cell of leukemic origin is CD70-positive, CD80-positive, and CD86-positive.

In certain exemplary embodiments, the modified cell of leukemic origin comprises an MHC class I molecule.

In certain exemplary embodiments, the modified cell of leukemic origin comprises an MHC class II molecule.

In certain exemplary embodiments, the modified cell of leukemic origin is non-proliferating.

In certain exemplary embodiments, the population of cells comprises PBMCs.

In certain exemplary embodiments, surface expression of CD25 and CD137 increases in natural killer cells after the co-culturing step.

Other embodiments will become apparent from a review of the ensuing detailed description, drawings and accompanying claims.

### BRIEF DESCRIPTION OF THE DRAWINGS

The foregoing and other features and advantages of the present disclosure will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 shows increased proliferation of NK cells within an allogeneic PBMC population when cocultured with DCOne-derived DCs, but not with monocyte-derived DCs, plus anti-PD-L1 antibodies with high Fc-receptor affinity (anti-PD-L1HA).

FIG. 2A - FIG. 2B show increased proliferation (2A) and frequency (2B) of NK cells within an allogeneic PBMC population when cocultured with DCOne-derived DCs plus anti-PD-L1 antibodies with high Fc-receptor affinity (anti-PD-L1HA).

FIG. 3 shows increased NK cell frequency and expansion within an allogeneic PBMC population when cocultured with DCOne-derived DCs plus anti-PD-L1 antibodies with high Fc-receptor affinity (anti-PD-L1HA).

FIG. 4 shows increased frequency and expansion of NK cells with memory-like phenotype within an allogeneic PBMC population when cocultured with DCOne-derived DCs plus anti-PD-L1 antibodies with high Fc-receptor affinity (anti-PD-L1HA) in PBMC/DCOne mDC and defucosylated CD16 high affinity anti-PD-L1 antibody co-cultures.

FIG. 5 shows enhanced PBMC cytotoxicity against K562 tumor cells induced by coculture of the PBMCs with DCOne-derived DCs plus anti-PD-L1 antibodies with high Fc-receptor affinity (anti-PD-L1HA).

FIG. 6 shows induced production of immune cell recruiting chemokines, and proinflammatory cytokines, including cytokines (IL-1 beta) derived from myeloid mononuclear cells, when allogeneic PBMCs are cocultured with DCOne-derived DCs plus anti-PD-L1 antibodies with high Fc-receptor affinity (anti-PD-L1HA).

### DETAILED DESCRIPTION

In one embodiment, methods for enhancing the pro-inflammatory function of FcγR-expressing immune cells in vivo are provided. Also provided herein are methods for improving the stimulation and expansion of immune cells (such as NK cells). In certain embodiments, the modified cell of leukemic origin is non-proliferating (e.g., has been irradiated). In certain embodiments, the non-proliferating modified cell of leukemic origin is an irradiated DCOne derived cell.

In another embodiment, methods of treating a disease or disorder are also provided, comprising the administration of a non-proliferating modified cell of leukemic origin and an anti-PD-L1 antibody in a subject receiving systemic treatment with adoptively transferred engineered immune cells or concomitantly treated with other immunotherapies, including immune checkpoint blockers. Such methods may enhance the efficacy or prolong the duration of the clinical effect of a genetically modified immune cell or immune checkpoint blocker. In certain embodiments, the modified cell of leukemic origin is non-proliferating (e.g., via irradiation). In certain embodiments, the non-proliferating modified cell of leukemic origin is a non-proliferating DCOne derived cell.

In certain embodiments, methods for ex vivo expansion of a population of cells (e.g., NK cells) is disclosed which comprise contacting a population of cells (e.g., NK cells) with a modified cell of leukemic origin and an anti-PD-L1 antibody to activate, stimulate and and/or expand the population of immune cells ex vivo.

It is to be understood that the methods described herein are not limited to particular methods and experimental conditions disclosed herein as such methods and conditions may vary. It is also to be understood that the terminology used herein is for the purpose of describing particular embodiments only, and is not intended to be limiting. The methods described herein use conventional molecular and cellular biological and immunological techniques that are well within the skill of the ordinary artisan. Such techniques are well known to the skilled artisan and are explained in the scientific literature.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer’s specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, e.g., ±5%, ±1%, or ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

The term “antigen” or “antigenic,” as used in relation to a polypeptide as described herein, refers generally to a biological molecule which contains at least one epitope specifically recognized by a T cell receptor, an antibody, or other elements of specific humoral and/or cellular immunity. The whole molecule may be recognized, or one or more portions of the molecule, for instance following intracellular processing of a polypeptide into an MHC peptide antigen complex and subsequent antigen presentation. The term “antigenic polypeptide” is interchangeable with “polypeptide antigen.” This terminology includes antigenic parts of said polypeptides, for instance produced after intracellular processing of a polypeptide and in the context of an MHC peptide antigen complex. The term “antigen” or “antigenic” includes reference to at least one, or more, antigenic epitopes of a polypeptide as described herein. In certain embodiments, a “non-tumor antigen” refers to herein as an antigen that is not derived from a tumor. For example, in certain embodiments, a non-tumor antigen may be a foreign antigen.

As used herein, the term “autologous” is meant to refer to any material derived from the same individual to which it is later to be re-introduced into the individual.

“Co-stimulatory ligand” refers to a molecule on an antigen presenting cell that specifically binds a cognate co-stimulatory molecule on an immune cell such as T cell, NK cell and/or NK-T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, MICB, CD155, CD112, HVEM, lymphotoxin beta receptor, 3/TR6, ILT3, ILT4, an agonist or antibody that binds Toll ligand receptor, and a ligand that specifically binds with B7-H3. A co-stimulatory ligand also encompasses, inter alia, an antibody that specifically binds with a co-stimulatory molecule present on an immune cell, such as but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on an immune cells that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, NKG2D, CD226, B7-H3, a ligand that specifically binds with CD83, and the like.

A “co-stimulatory signal,” as used herein, refers to a signal, which in combination with a primary signal, such as TCR/CD3 ligation, in case of T cell leads to proliferation and/or upregulation or downregulation of key molecules. In certain exemplary embodiments, the co-stimulatory signal is CD70.

A “disease” is a state of health of an animal wherein the animal cannot maintain homeostasis, and wherein if the disease is not ameliorated then the animal’s health continues to deteriorate. In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of immune cells such as NK cells. In one embodiment, the NK cells that are expanded ex vivo increase in number relative to the number originally present in the culture. In another embodiment, the NK cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

An “NK cell” as used herein generally refers to a CD56+CD3- lymphoid cell, which is further subdivided into two major subpopulations based on CD56 and CD16 receptor surface expression: CD56dimCD16bright and CD56brightCD16dim cells. Circulating CD56dimCD16bright NK cells are quiescent but become highly cytotoxic upon recognition of target cells, CD56brightCD16dim cells, that reside in secondary lymphoid tissues, constitutively produce cytokines.

A “modified NK cell” as used herein refers to an NK cell that has been engineered, e.g., genetically engineered. In certain exemplary embodiments, a modified NK cell described herein may have one or any combination of improved cytotoxicity (e.g., by exogenous expression of chimeric antigen receptors (CARs) and activating receptors, or selective downregulation of inhibitory receptors), reduced sensitivity to the tumor microenvironment (e.g., by downregulation of inhibitory cytokines and/or small molecule receptors, the introduction of dominant-negative receptors, etc.), increased in vivo proliferation and persistence via autocrine cytokines stimulation and tumor homing (e.g., by the expression of chemokine receptors).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

The term “immune response,” as used herein, includes NK cell and myeloid mononuclear cell mediated immune responses. Exemplary immune functions of NK cells include, e.g., cytokine production and induction of cytotoxicity in other cells. Myeloid mononuclear cell (monocytes, macrophages and dendritic cells) include cytokine (including IL-1 beta) and chemokine production. In addition, the term includes immune responses that are indirectly affected by NK cell and myeloid mononuclear cell activation, e.g., tumor-antigen loading and activation of “bystander” dendritic cells Immune cells involved in the immune response also include lymphocytes, such as B cells and T cells (CD4⁺ and CD8⁺ cells); Langerhans cells, and non-professional antigen presenting cells such as granulocytes, keratinocytes, endothelial cells, astrocytes, fibroblasts, oligodendrocytes. In certain embodiments, the term refers to a T cell mediated immune response. The immune response may in some embodiments be a T cell-dependent immune response.

The term “T cell dependent immune response,” as used herein, refers to an immune response wherein either T cells, B cells or both T cell and B cell populations are activated, and wherein T cells further assist T and B cells and other immune cells in executing their function.

The term “immunosuppressive” is used herein to refer to reducing overall immune response.

“Insertion/deletion,” commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, e.g., a human.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., intratumoral, intradermal, subcutaneous (s.c.), intravenous (i.v.), intramuscular (i.m.), or intraosseous injection, or infusion techniques.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a Fc-receptor) with its cognate ligand thereby (Fc-domain of IgG) mediating a signal transduction event.

The term “subject,” as used herein, refers to the recipient of a method as described herein, i.e., a recipient that can mount a cellular immune response, and is a mammal. In certain embodiments, the subject is a human. In certain embodiments, the subject is a domesticated animal, e.g., a horse, a cow, a pig, a sheep, a dog, a cat, etc. The terms “patient” and “subject” may be used interchangeably. In certain embodiments, the subject is a human suffering from a tumor (e.g., a solid tumor). In certain embodiments, the subject is a domesticated animal suffering from a tumor (e.g., a solid tumor).

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The term “tumor,” as used herein, includes reference to cellular material, e.g., a tissue, proliferating at an abnormally high rate. A growth comprising neoplastic cells is a neoplasm, also known as a “tumor,” and generally forms a distinct tissue mass in a body of a subject. A tumor may show partial or total lack of structural organization and functional coordination with the normal tissue. As used herein, a tumor is intended to encompass hematopoietic tumors as well as solid tumors. In certain embodiments, the tumor is a solid tumor. The term “tumor,” as used herein, includes reference to the tumor micro-environment or tumor site, i.e., the area within the tumor and the area directly outside the tumorous tissue. In certain embodiments, the tumor micro-environment or tumor site includes an area within the boundaries of the tumor tissue. In certain embodiments, the tumor micro-environment or tumor site includes the tumor interstitial compartment of a tumor, which is defined herein as all that is interposed between the plasma membrane of neoplastic cells and the vascular wall of the newly formed neovessels. As used herein, the terms “tumor micro-environment” or “tumor site” refers to a location within a subject in which a tumor resides, including the area immediately surrounding the tumor.

A tumor may be benign (e.g., a benign tumor) or malignant (e.g., a malignant tumor or cancer). Malignant tumors can be broadly classified into three major types: those arising from epithelial structures are called carcinomas, those that originate from connective tissues such as muscle, cartilage, fat or bone are called sarcomas, and those affecting hematopoietic structures (structures pertaining to the formation of blood cells) including components of the immune system, are called leukemias and lymphomas. Other tumors include, but are not limited to, neurofibromatosis. In certain exemplary embodiments, the tumor is a glioblastoma. In certain exemplary embodiments, the tumor is an ovarian cancer (e.g., an epithelial ovarian cancer, which can be further subtyped into a serous, a clear cell, an endometrioid, a mucinous, or a mixed epithelial ovarian cancer).

Solid tumors are abnormal masses of tissue that can be benign or malignant. In certain embodiments, solid tumors are named for the type of cells that form them (such as sarcomas, carcinomas, and lymphomas). Examples of solid tumors, such as sarcomas and carcinomas, include, but are not limited to, liposarcoma, fibrosarcoma, chondrosarcoma, osteosarcoma, myxosarcoma, and other sarcomas, mesothelioma, synovioma, leiomyosarcoma, Ewing’s tumor, colon carcinoma, rhabdomyosarcoma, pancreatic cancer, lymphoid malignancy, lung cancers, breast cancer, prostate cancer, ovarian cancer, hepatocellular carcinoma, adenocarcinoma, basal cell carcinoma, sweat gland carcinoma, squamous cell carcinoma, medullary thyroid carcinoma, pheochromocytomas sebaceous gland carcinoma, papillary thyroid carcinoma, papillary adenocarcinomas, papillary carcinoma, medullary carcinoma, bronchogenic carcinoma, hepatoma, renal cell carcinoma, bile duct carcinoma, Wilms’ tumor, choriocarcinoma, cervical cancer, seminoma, testicular tumor, bladder carcinoma, melanoma, CNS tumors (e.g., a glioma, e.g., brainstem glioma and mixed gliomas, glioblastoma (e.g., glioblastoma multiforme), germinoma, astrocytoma, craniopharyngioma, medulloblastoma, ependymoma, Schwannoma, CNS lymphoma, acoustic neuroma, pinealoma, hemangioblastoma, meningioma, oligodendroglioma, retinoblastoma, neuroblastoma, and brain metastases), and the like.

Carcinomas that can be amenable to therapy by a method disclosed herein include, but are not limited to, squamous cell carcinoma (various tissues), basal cell carcinoma (a form of skin cancer), esophageal carcinoma, bladder carcinoma, including transitional cell carcinoma (a malignant neoplasm of the bladder), hepatocellular carcinoma, colorectal carcinoma, bronchogenic carcinoma, lung carcinoma, including small cell carcinoma and non-small cell carcinoma of the lung, colon carcinoma, thyroid carcinoma, gastric carcinoma, breast carcinoma, ovarian carcinoma, adrenocortical carcinoma, pancreatic carcinoma, sweat gland carcinoma, prostate carcinoma, papillary carcinoma, adenocarcinoma, sebaceous gland carcinoma, medullary carcinoma, papillary adenocarcinoma, ductal carcinoma in situ or bile duct carcinoma, cystadenocarcinoma, renal cell carcinoma, choriocarcinoma, Wilm’s tumor, seminoma, embryonal carcinoma, cervical carcinoma, testicular carcinoma, nasopharyngeal carcinoma, osteogenic carcinoma, epithelial carcinoma, uterine carcinoma, and the like.

Sarcomas that can be amenable to therapy by a method disclosed herein include, but are not limited to, myxosarcoma, chondrosarcoma, chordoma, osteogenic sarcoma, liposarcoma, fibrosarcoma, angiosarcoma, lymphangiosarcoma, endotheliosarcoma, osteosarcoma, mesothelioma, Ewing’s sarcoma, leiomyosarcoma, rhabdomyosarcoma, lymphangioendotheliosarcoma, synovioma, and other soft tissue sarcomas.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

The term “immunogenic composition,” as used herein, refers to a substance which induces a specific immune response against an immunogen in a subject who is in need of an immune response against said immunogen. The composition may include an adjuvant and optionally one or more pharmaceutically-acceptable carriers, excipients and/or diluents. The immunogenic composition can be employed in prime-boost vaccination, such as at least 2, 3, 4 or at least 5 immunizations separated in time. The immunogenic composition can be an (allogeneic) dendritic cell comprising said immunogen.

As used herein, the term “extratumoral” refers to a location, e.g., in the body of a subject, that is away (e.g., distal) from a tumor and immediately surrounding tissue (e.g., that may make up the tumor micro-environment).

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Cell of Leukemic Origin

Provided herein are methods comprising the use of a modified cell of leukemic origin for methods of treatment. The term “modified cell of leukemic origin,” as used herein, refers to a cell that can take up an antigen such as an antigenic polypeptide into its cell, and presents the antigen, or an immunogenic part thereof together with an MHC class I complex or MHC class II complex. In certain embodiments, the modified cell of leukemic origin is a cell derived from cell line DCOne as deposited under the conditions of the Budapest treaty with the DSMZ under accession number DSMZ ACC3189 on 15 Nov. 2012. The process of obtaining mature cells from the deposited DCOne cell line is, for instance, described in EP2931878B1. The term “dendritic cell,” as used herein, refers to a professional antigen presenting cell (APC) that can take up an antigen such as an antigenic polypeptide into its cell, and presents the antigen, or an immunogenic part thereof together with an MHC class I complex or MHC class II complex. Having a mature dendritic cell phenotype means that the modified cell of leukemic origin is capable of performing similar functions to those of a mature dendritic cell. The term includes both immature dendritic cells (“imDC”) and mature dendritic cells (“mDC”), depending on maturity.

In certain embodiments, the modified cell of leukemic origin is derived from leukemia cells. In certain embodiments, the modified cell of leukemic origin is derived from a patient having leukemia. In certain embodiments, the modified cell of leukemic origin is derived from the peripheral blood of a patient having leukemia. In certain embodiments, the modified cell of leukemic origin is derived from the peripheral blood of a patient having acute myeloid leukemia. The skilled artisan will recognize that a modified cell of leukemic origin can be derived from any patient obtained peripheral blood, wherein the patient has any type of leukemia, given that the modified cell of leukemic origin thus derived comprises the characteristics disclosed herein.

In certain embodiments, the modified cell of leukemic origin is CD34-positive, CD1a-positive, and CD83-positive. In certain embodiments, the modified cell of leukemic origin comprises a cell surface marker selected from the group consisting of CD14, DC-SIGN, Langerin, CD40, CD70, CD80, CD83, CD86, and any combination thereof. In certain embodiments, the modified cell of leukemic origin comprises an MHC class I molecule. In certain embodiments, the modified cell of leukemic origin comprises an MHC class II molecule. In certain embodiments, the modified cell of leukemic origin is CD34-positive, CD1a-positive, CD83-positive, and CD14-negative. In certain embodiments, the modified cell of leukemic origin is CD40-positive, CD80-positive, and CD86-positive. In certain embodiments, the modified cell of leukemic origin is CD34-positive, CD1a-positive, CD83-positive, CD40-positive, CD80-positive, CD86-positive, and CD14-negative.

In certain embodiments, the modified cell of leukemic origin comprises a genetic aberration between chromosome 11p15.5 to 11p12. In certain embodiments, the genetic aberration encompasses about 16 Mb of genomic regions (e.g., from about 20.7 Mb to about 36.6 Mb). In certain embodiments, the genetic aberration contains a loss of about 60 known and unknown genes.

In certain embodiments, the modified cell of leukemic origin comprises a co-stimulatory molecule. In certain embodiments, the co-stimulatory molecule includes, without limitation, an MHC class I molecule, BTLA and Toll ligand receptor. Examples of co-stimulatory molecules include CD70, CD80, CD86, 4-1BBL (CD137-ligand), OX40L, CD30L, CD40, PD-L1, ICOSL, ICAM-1, lymphocyte function-associated antigen 3 (LFA3 (CD58)), K12/SECTM1, LIGHT, HLA-E, B7-H3 and CD83.

In certain embodiments, the modified cell of leukemic origin comprises at least one endogenous antigen. Depending on the leukemic origin of the modified cell, the modified cell of leukemic origin may comprise at least one known endogenous antigen that is specific to the leukemic origin. In certain embodiments, the endogenous antigen is a tumor-associated antigen. In certain embodiments, an endogenous tumor-associated antigen may be selected from the group consisting of WT-1, RHAMM, PRAME, p53, Survivin, and MUC-1.

In certain embodiments, the modified cell of leukemic origin is a cell of cell line DCOne as described in PCT Publication Nos. WO 2014/006058 and WO 2014/090795, the disclosures of which are incorporated by reference herein in their entireties. In certain embodiments, modified cell of leukemic origin is a cell of cell line DCOne and comprises a mature dendritic cell phenotype that is CD34-positive, CD1a-positive, and CD83-positive. In certain embodiments, modified cell of leukemic origin is a cell of cell line DCOne and is CD34-positive, CD1a-positive, and CD83-positive. In certain embodiments, modified cell of leukemic origin is a cell of cell line DCOne and comprises a cell surface marker selected from the group consisting of CD14, DC-SIGN, Langerin, CD80, CD86, CD40, CD70, and any combination thereof. In certain embodiments, modified cell of leukemic origin is a cell of cell line DCOne and comprises MHC class I. In certain embodiments, modified cell of leukemic origin is a cell of cell line DCOne and comprises MHC class II. In certain embodiments, the modified cell of leukemic origin is a cell of cell line DCOne and is CD34-positive, CD1a-positive, CD83-positive, and CD14-negative. In certain embodiments, the modified cell of leukemic origin is a cell of cell line DCOne and is CD40-positive, CD80-positive, and CD86-positive. In certain embodiments, the modified cell of leukemic origin is a cell of cell line DCOne and is CD34-positive, CD1a-positive, CD83-positive, CD40-positive, CD80-positive, CD86-positive, and CD14-negative. In certain embodiments, modified cell of leukemic origin is a cell of cell line DCOne and comprises a genetic aberration between chromosome 11p15.5 to 11p12. In certain embodiments, modified cell of leukemic origin is a cell of cell line DCOne and comprises a genetic aberration that encompasses about 16 Mb of genomic regions (e.g., from about 20.7 Mb to about 36.6 Mb). In certain embodiments, modified cell of leukemic origin is a cell of cell line DCOne and comprises a genetic aberration that contains a loss of about 60 known and unknown genes.

As provided herein, certain methods are directed to the use of a modified cell of leukemic origin, wherein the modified cell is non-proliferating. In certain embodiments, the modified cell of leukemic origin has been irradiated. In certain embodiments, the modified cell of leukemic origin has been irradiated prior to its use in a method disclosed herein. Irradiation can, for example, be achieved by gamma irradiation at 30 - 150 Gy, e.g., 100 Gy, for a period of 1 to 3 hours, using a standard irradiation device (Gammacell or equivalent). Irradiation ensures that any remaining progenitor cell in a composition comprising the modified cell of leukemic origin, e.g., a CD34 positive cell, cannot continue dividing. The cells may, for example, be irradiated prior to injection into patients, when used as a vaccine, or immediately after cultivating is stopped. In certain embodiments, the cells are irradiated to inhibit their capacity to proliferate and/or expand, while maintaining their immune stimulatory capacity.

### C. Methods of Treatment

In certain exemplary embodiments, methods of using modified cells of leukemic origin together with an anti-PD-L1 antibody in an adoptive cell therapy for treating a disease or disorder in a subject are provided. In one aspect, a method for treating a disease or disorder in a subject, comprising: administering to the subject a composition comprising a modified cell of leukemic origin and anti-PD-L1 antibodies with high affinity to Fc-receptors; and administering to the subject an adoptive cell therapy. Adoptive cell therapy is an immunotherapy in which immune cells (e.g., T cells and NK cells) are given to a subject to fight diseases, such as cancer, is provided. In general, T cells can be obtained from the subject’s own peripheral blood or tumor tissue, stimulated and expanded ex vivo, and then administered back to the subject (i.e., autologous adaptive cell therapy. In certain embodiments, T cells can be obtained from a first subject (e.g., from peripheral blood or tumor tissue of the first subject), stimulated and expanded ex vivo, and then administered to a second subject (i.e., allogeneic adaptive cell therapy).

In certain embodiments, the T cells and NK cells can be modified ex vivo (e.g., genetically modified) to express an immune receptor (e.g., a TCR and/or CAR). The term “adoptive cell therapy” refers to both T cell and NK cell therapy without genetic modification, and T cell and NK cell therapy with genetic modification to, e.g., express an immune receptor.

As such, in certain embodiments, provided herein is a method for treating a disease or disorder in a subject in need thereof, comprising: administering to the subject a first composition comprising a modified cell of leukemic origin and an anti-PD-L1 antibody; and administering to the subject a second composition comprising a modified immune cell, wherein the modified immune cell comprises an immune receptor. In certain embodiments, the immune receptor is a TCR and/or CAR as described elsewhere herein.

In certain embodiments, the disease or disorder is a cancer. In certain embodiments, the cancer is a tumor. In certain embodiments, the cancer is a semi-solid tumor, or a solid tumor.

### D. Pharmaceutical Compositions and Formulations

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In certain embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In certain embodiments, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In certain embodiments, a mixture of two or more preservatives is used. The preservative or mixtures thereof are typically present in an amount of about 0.0001 % to about 2% by weight of the total composition. Carriers are described, e.g., by Remington’s Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in certain embodiments are included in the compositions. Suitable buffering agents include, for example, citric acid, sodium citrate, phosphoric acid, potassium phosphate, and various other acids and salts. In certain embodiments, a mixture of two or more buffering agents is used. The buffering agent or mixtures thereof are typically present in an amount of about 0.001 % to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, e.g., those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In certain embodiments, the cell populations are administered parenterally. The term “parenteral,” as used herein, includes intravenous, intramuscular, subcutaneous, rectal, vaginal, and intraperitoneal administration. In certain embodiments, the cells are administered to the subject using peripheral systemic delivery by intravenous, intraperitoneal, or subcutaneous injection. Compositions in certain embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### E. Experimental Examples

The following examples are put forth so as to provide those of ordinary skill in the art with a complete disclosure and description of how to make and use the methods and compositions described herein, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Increased Proliferation and Frequency of NK Cells Within an Allogeneic PBMC Population When Cocultured With DCOne-derived DCs plus anti-PD-L1 Antibodies with High Fc-receptor Affinity (anti-PD-L1HA)

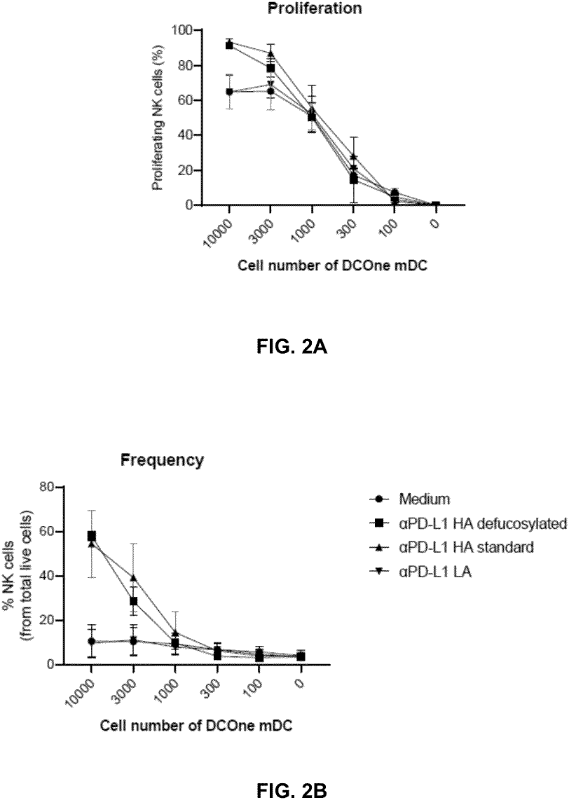

FIG. 2 shows Increased proliferation and frequency of NK cells in PBMC in a MLR assay. CSFE-labelled PBMC were co-cultured with varying numbers of DCOne mDC for six days in the absence or presence of different class of anti-PD-L1 antibodies, i.e., non-glycosylated antibody with low affinity for CD16 and glycosylated antibodies with high affinity for CD16. The latter further distinguished into standard (normal glycosylated) and defucosylated antibodies. The three anti-PD-L1 variants αPDL1 LA (inverted triangles), αPDL1 HA standard (triangles), and αPDL1 HA defucosylated (black squares). Medium (circles) culture in the absence of antibodies served as a negative control. The proliferation of CFSE-labelled NK cells in the mixed lymphocyte reaction (MLR) was determined on day 6 by CFSE dilution measured by flow cytometric analysis. The standard (normal glycosylated) and defucosylated antibodies with high affinity for CD16 had similar performance, both performing better than the anti-PD-L1 antibodies with low affinity for CD16.

### Example 2. Increased Proliferation of NK Cells Within an Allogeneic PBMC Population When Cocultured With DCOne-derived DCs, but not With Monocyte-Derived DCs, Plus anti-PD-L1 Antibodies With High Fc-receptor Affinity (anti-PD-L1 HA)

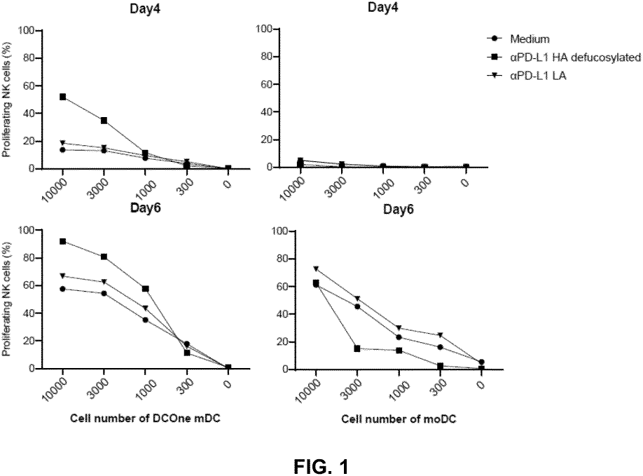

CSFE-labelled PBMC were co-cultured with varying numbers of DCOne mDC (upper and lower left graphs) and monocyte-derived DC (moDC; upper and lower right graphs) in the absence or presence of different class of anti-PD-L1 antibodies, i.e., non-glycosylated antibody with low affinity for CD16 receptor αPDL1 LA (inverted triangles) and defucosylated antibody with high affinity for CD16 receptor αPDL1 HA defucosylated (black squares). Medium (circles) culture in the absence of antibodies served as a negative control. At day 4 and day 6 cells were harvested and stained with anti-CD56 specific antibodies and CFSE dilution in CD56 + ve NK cells was analyzed by flow cytometry as measure of proliferation. FIG. 1 shows that NK cell proliferation increased in PBMC-DCOne mDC co-culture as compared to PBMC-monocyte-derived DC co-culture in anti-PD-L1 antibodies that have high affinity for CD16 (a.k.a., CD16 high affinity anti-PD-L1 antibodies).

### Example 3. Increased NK Cell Frequency and Expansion Within an Allogeneic PBMC Population When Cocultured With DCOne-derived DCs plus anti-PD-L1 Antibodies With High Fc-receptor Affinity (anti-PD-L1HA)

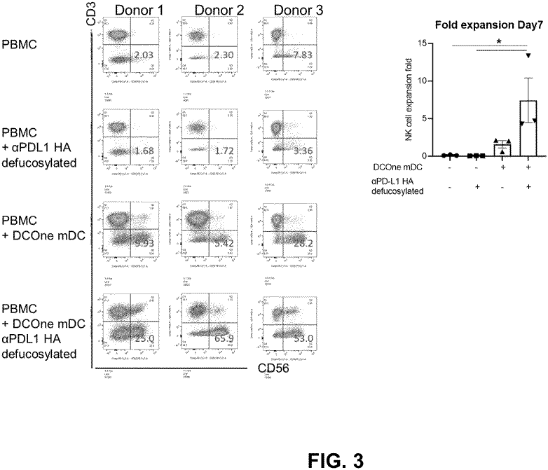

FIG. 3 shows the frequency of total NK cells on day 7 co-cultures of PBMC without and with either DCOne mDC, defucosylated CD16 high affinity anti-PD-L1 antibody or combination of the two in a MLR assay. At day 7 cells were harvested and stained with anti-CD56, CD3 specific antibodies and analyzed by flow cytometry. Cell counts of different groups were 0.13, 0.06, 1.58 and 7.44 as fold increase, respectively, as shown in FIG. 3, right panel. These results demonstrate increased NK cell frequency and expansion in PBMC/DCOne mDC and defucosylated CD16 high affinity anti-PD-L1 antibody.

### Example 4. Increased Frequency and Expansion of NK Cells With Memory-like Phenotype Within an Allogeneic PBMC Population When Cocultured With DCOne-derived DCs Plus anti-PD-L1 Antibodies With High Fc-receptor Affinity (anti-PD-L1 HA)

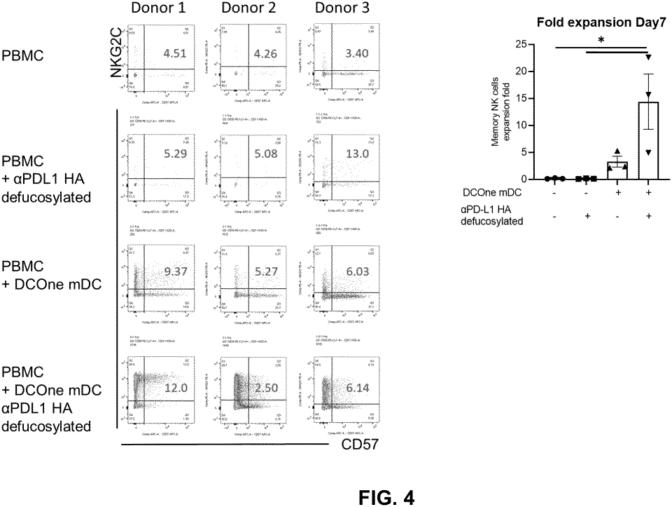

FIG. 4 shows the frequency of total NK cells on day 6 co-cultures of PBMC without and with either DCOne mDC, defucosylated CD16 high affinity anti-PD-L1 antibody or combination of the two in a MLR assay. At day 6 cells were harvested and stained with anti-CD56, CD3, NKG2C and CD57 specific antibodies and analyzed by flow cytometry. Cell counts of different groups were 0.18, 0.15, 4.83 and 34.86 as fold increase, respectively, as shown in FIG. 4, right panel.

### Example 5. Enhanced PBMC Cytotoxicity Against K562 Tumor Cells Induced by Coculture Of the PBMCs With DCOne-derived DCs Plus anti-PD-L1 Antibodies With High Fc-receptor Affinity (anti-PD-L1HA)

To assay tumor cell killing, tumor cell line K562 were incubated with NK cells from 6 day co-cultures. The killing of tumor cells by activated NK cells were evaluated after 60 minutes of incubation time using the GranToxiLux assay (Oncolmmunin). This assay visualized the active amount of the cytolytic enzyme Granzyme B (GrzB) inside the tumor cells; and the binding of a fluorochrome-labelled substrate (TFL4) to active GrzB in tumor cells is visualized by flow cytometry.

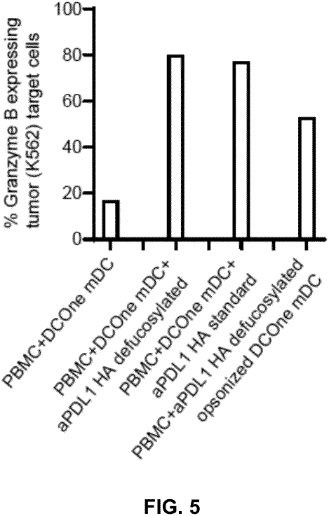

Tumor cell line K562 was labeled with fluorescent cell linker dye TFL4 and co-incubated with NK cells from different cocultures for 1 hour at an effector : target ratio of 10:1 in the presence of fluorogenic granzyme B substrate. As shown in FIG. 5, co-incubation with activated NK cells resulted in increased detection of fluorescence in the tumor, as detected by multiparameter flow cytometry. Fluorogenic Granzyme B activity in the target tumor cells after cleavage of the granzyme B substrate was measured by using the GRANTOXILUX™ kit (Oncolmmunin, Inc., MD).

### Example 6. Induced Production of Immune Cell Recruiting Chemokines And Proinflammatory Cytokines When Allogeneic PBMCs are Cocultured With DCOne-derived DCs Plus anti-PD-L1 Antibodies with High Fc-receptor Affinity (anti-PD-L1HA)

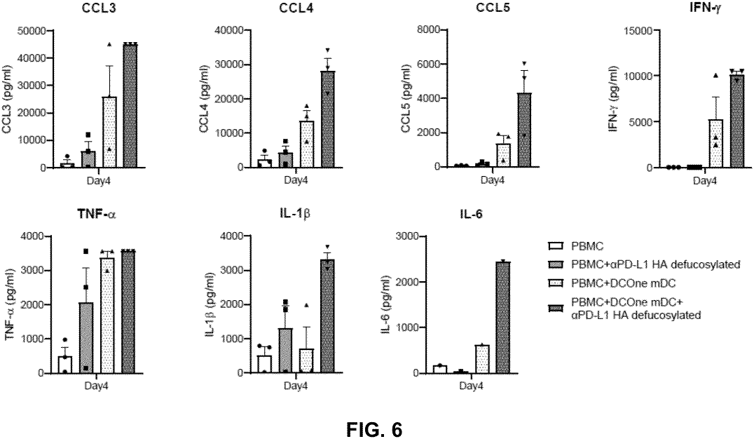

PBMC were co-cultured with DCOne mDC (at 1: 10 DCOne mDC:PBMC ratio) in the absence or presence of defucosylated antibody with high affinity for CD16 receptor αPDL1 HA defucosylated for 4 days in 24-wells plate. Medium culture in the absence of antibodies served as a negative control. At day 4 supernatants were harvested and analyzed for secreted chemokines and cytokines using magnetic-bead arrays using Luminex platform (FIG. 6).

### Example 7. DCOne-derived Mature DCs Opsonized With anti-PD-L1 Antibodies As Intratumoral Immune Primers

### Introduction

One factor associated with the outcome of immune checkpoint blockade (ICB) therapy is whether the tumors are “hot” or “cold”. In a recent study an essential role for functional Fc-gamma-receptors (FcyR) on intratumoral NK cells and macrophages in the induction of a “hot” tumor was shown.

Mature dendritic cells derived from the leukemic cell line DCOne (DCOne mDC) strongly activate cocultured allogeneic T cells, while expressing PD-L1. It was investigated how opsonization of these cells with anti-PD-L1 antibodies could affect the activation of FcyR expressing NK cells and monocytes within cocultured allogeneic PBMCs.

### Material and Methods

PBMCs from healthy CMV+ donors were co-cultured with DCOne mDCs +/- different anti-PD-L1 antibodies for 4-7 days without stimulating cytokines. Proliferation (CFSE), cytotoxicity (granzyme B; GrB) and immune cell phenotype was monitored with flow cytometry and cytokine/chemokine production with Luminex.

### Results

Addition of anti-PD-L1 antibodies with high FcγR affinity, but not low affinity antibodies, led to prominent NK cell activation (CD25 expression). When analyzing immune cell phenotypes after 7 days co-culture the relative frequency of CD56+CD3- NK cells among cocultured PBMCs was dramatically increased, particularly the subgroup of NK cells co-expressing the adaptive/memory markers NKG2C and CD57. As to functionality, the tumor killing capacity (GrB-expressing K562 target cells) was strongly enhanced. Finally, addition of anti-PD-L1 to the mDC/PBMC co-culture led to strongly enhanced production of several proinflammatory cytokines, including TNF-α and IFN-γ, and chemokines, including CCL-3, 4, and 5. Notably, the production of IL-1β was by far highest when anti-PD-L1 antibodies were present, indicating an Fc-receptor-mediated activation of monocytes.

### Conclusion

Taken together, these data indicate that DCOne mDCs opsonized with anti-PD-L1 antibodies with high Fc-receptor affinity induced a strong activation of co-cultured NK cells and monocytes from CMV+ donors. Without intending to be bound by scientific theory, if administered intratumorally, these opsonized “off-the-shelf” leukemia-derived mDCs could induce Fc-gamma-receptor dependent activation of intratumoral macrophages and NK cells (as well as activation of alloreactive T cells) that could initiate critical steps including local tumor cell killing and recruitment of immune cells, including cross-presenting DCs, and that could sensitize for concomitant ICB therapy.

What is claimed is:

1. A composition comprising
a modified cell of leukemic origin, and
an anti-PD-L1 antibody,
wherein the modified cell exhibits a mature dendritic cell phenotype.

2. The composition of claim 1, wherein the modified cell of leukemic origin comprises at least one tumor antigen selected from the group consisting of WT-1, RHAMM, PRAME, MUC-1, p53, and Survivin.

3. The composition of claim 1, wherein the modified cell of leukemic origin is CD34-positive, CD1a-positive, CD83-positive, and CD14-negative.

4. The composition of claim 1, wherein the anti-PD-L1 antibody comprises an IgG domain that binds with high affinity to Fc-gamma receptors (FcgRs), including FcgRI (CD64), FcgRII (CD32) and FcgRIIIA (CD16).

5. The composition of claim 1, further comprising a plurality of peripheral blood mononuclear cells (PBMCs), natural killer (NK) cell line cells, cord blood stem cells, pluripotent stem cells and any combination thereof.

6. The composition of claim 5, wherein a plurality of allogeneic NK cells is derived from the PBMCs, the NK cell line cells, the cord blood stem cells, the pluripotent stem cells and the combination thereof.

7. The composition of claim 5, wherein the PBMCs are derived from a patient to treat.

8. The composition of claim 5,
wherein the modified cell of leukemic origin and the anti-PD-L1 antibody have a synergistic effect on activating FCgR-expressing NK cells and myeloid mononuclear cells;
wherein the activation of the FCgR-expressing NK cells and myeloid mononuclear cells results in increased secretion of chemokines and proinflammatory cytokines; and/or
wherein the activation of the NK cells results in increased NK-mediated lysis of tumor cells.

9. (canceled)

10. (canceled)

11. The composition of claim 1, wherein the anti-PD-L1 antibody is selected from the group consisting of an IgG Fc domain with high affinity to FcgRs, that is optionally avelumab or PDL-GEX.

12. The composition of claim 1, wherein the modified cell of leukemic origin further comprises a cell surface marker selected from the group consisting of DC-SIGN, Langerin, CD40, CD70, CD80, CD86, and any combination thereof;
wherein the modified cell of leukemic origin is CD70-positive, CD80-positive, and CD86-positive;
wherein the modified cell of leukemic origin comprises an MHC class I molecule;
wherein the modified cell of leukemic origin comprises an MHC class II molecule; and/or
wherein the modified cell of leukemic origin is non-proliferating.

13-16. (canceled)

17. A method for activating, stimulating and and/or expanding a population of immune cells, comprising:
(a) obtaining a population of cells comprising immune cells;
(b) contacting the population of cells with a modified cell of leukemic origin and an anti-PD-L1 antibody, wherein the modified cell exhibits a mature dendritic cell phenotype; and
(c) co-culturing the population of cells and the modified cell of leukemic origin and the anti-PD-L1 antibody under conditions suitable to induce activation of the immune cells, thereby expanding the population of immune cells.

18. The method of claim 17, wherein:
the modified cell of leukemic origin comprises at least one tumor antigen selected from the group consisting of WT-1, RHAMM, PRAME, MUC-1, p53, and Survivin;
the modified cell of leukemic origin is CD34-positive, CD1a-positive, CD83-positive, and CD14-negative;
the modified cell of leukemic origin further comprises a cell surface marker selected from the group consisting of DC-SIGN, Langerin, CD40, CD70, CD80, CD86, and any combination thereof;
the modified cell of leukemic origin is CD70-positive, CD80-positive, and CD86-positive;
the modified cell of leukemic origin comprises an MHC class I molecule;
the modified cell of leukemic origin comprises an MHC class II molecule;
the modified cell of leukemic origin is non-proliferating;
the population of cells comprises PBMCs; and/or
surface expression of CD25 and CD137 increases in natural killer cells after the co-culturing step.

19-26. (canceled)

27. A method for treating a disease or disorder in a subject in need thereof, comprising
administering intratumorally to the subject a first composition comprising a modified cell of leukemic origin and an anti-PD-L1 antibody, wherein the modified cell exhibits a mature dendritic cell phenotype, optionally wherein the anti-PD-L1 antibody is a bispecific or multi-specific antibody; or
A method for treating a disease or disorder in a subject in need thereof, comprising
administering intratumorally to the subject a composition comprising:
a modified cell of leukemic origin, wherein the modified cell exhibits a mature dendritic cell phenotype;
anti-PD-L1 antibody; and
autologous or allogeneic PBMCs.

28. (canceled)

29. The method of claim 27,
wherein the modified cell of leukemic origin:
comprises at least one tumor antigen selected from the group consisting of WT-1, RHAMM, PRAME, MUC-1, p53, and Survivin;
is CD34-positive, CD1a-positive, CD83-positive, and CD14-negative;
further comprises a cell surface marker selected from the group consisting of DC-SIGN, Langerin, CD40, CD70, CD80, CD86, and any combination thereof;
is CD70-positive, CD80-positive, and CD86-positive;
comprises an MHC class I molecule;
comprises an MHC class II molecule; and/or
is non-proliferating; and/or
wherein the anti-PD-L1 antibody is a bispecific or multi-specific antibody.

30-36. (canceled)

37. The method of claim 27, wherein surface expression of CD25 and CD137 both increases in the natural killer cells after being administered to the subject.

38. The method of claim 27, wherein the disease or disorder is a cancer, optionally wherein the cancer is a semi-solid tumor, a solid tumor, acute myeloid leukemia (AML), or lymphoma.

39-41. (canceled)

42.
1) A method of making a composition, comprising adding an anti-PD-L1 antibody to a modified cell of leukemic origin, wherein the modified cell of leukemic origin exhibits a mature dendritic cell phenotype, optionally wherein the anti-PD-L1 antibody is a bispecific or multi-specific antibody; or
2) A composition comprising ex vivo expanded immune cells, comprising a population of immune cells, a modified cell of leukemic origin, and an anti-PD-L1 antibody, wherein the modified cell exhibits a mature dendritic cell phenotype.

43. The composition claim 1, wherein the anti-PD-L1 antibody is a bispecific or multi-specific antibody.

44. The method of claim 17, wherein the anti-PD-L1 antibody is a bispecific or multi-specific antibody.

45. (canceled)

46. The composition of claim 42, wherein:
the modified cell of leukemic origin comprises at least one tumor antigen selected from the group consisting of WT-1, RHAMM, PRAME, MUC-1, p53, and Survivin;
the modified cell of leukemic origin is CD34-positive, CD1a-positive, CD83-positive, and CD14-negative,
the modified cell of leukemic origin further comprises a cell surface marker selected from the group consisting of DC-SIGN, Langerin, CD40, CD70, CD80, CD86, and any combination thereof;
the modified cell of leukemic origin is CD70-positive, CD80-positive, and CD86-positive;
the modified cell of leukemic origin comprises an MHC class I molecule;
the modified cell of leukemic origin comprises an MHC class II molecule;
the modified cell of leukemic origin is non-proliferating;
the population of immune cells comprises PBMCs; and/or
surface expression of CD25 and CD137 increases in natural killer cells after the co-culturing step.

47-54. (canceled)
